(19) United States
(12) Patent Application Publication
(10) Pub. No.: US 2003/0073085 A1
(43) Pub. Date: Apr. 17, 2003

(54) Amplifying expressed sequences from genomic DNA of higher-order eukaryotic organisms for DNA arrays

(76) Inventors: Fang Lai, Painted Post, NY (US); Daixing Zhou, Horseheads, NY (US)

Correspondence Address: CORNING INCORPORATED, SP-TI-3-1, CORNING, NY, 14831

(21) Appl. No.: 09/972,469

(22) Filed: Oct. 5, 2001

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

A method for amplifying expressed sequences from genomic DNA (gDNA) selected from a mammalian or higher order plant species using the 3′UTR of the gene sequence. 3′UTR typically exists as a single exon. A 3′UTR of a gDNA sequence or an exon of a gene defined by computer software is identified based on the presence of a stop codon and a polyadenylation signal in the gDNA sequence corresponding to a expressed mRNA sequence. A gDNA sequence that is highly unique to the given gene is selected, and a probe for the sequence is designed. Two rounds of polymerase chain reaction are performed on the 3′UTR sequence. PCR product from the first round is separated by size-differentiation, and a predetermined band from the size-differentiated samples is chosen. Without need for purification, a second round of PCR is performed to amplify the predetermined sequence of gDNA. The method provides alternative process to acquire and amplify expressed sequences, especially for those which cDNA clones are not available. Hence, the method is useful in fabricating high-density DNA arrays of enhanced, widely varying genetic content.

### FIELD OF THE INVENTION

The present invention relates to a method and devices that embody the method for in vitro amplification of expressed sequences directly from genomic DNA (gDNA) of all mammalian and/or higher-order plant species for DNA array fabrication. The method can be used to selectively amplify nucleic acid sequences, which contain sequence variations such as point mutations, deletions and insertions.

### BACKGROUND

High-density arrays (HDAs) of cDNA or oligonucleotide have been powerful tools for profiling gene expression of particular cell or tissue types. Researchers have employed HDAs in their studies to uncover relationships between known genes, as well as, to reveal the function of previously uncharacterized genes. In current HDAs, the expressed genetic sequences, which are printed on the solid surfaces that form the arrays, typically come in two basic forms, selected from either 1) DNA fragments amplified from cDNA clones or genomic DNA of single cell organisms, or 2) synthetic oligonucleotides.

The current technology, while useful, has many associated problems, in particular regarding the amplification of cDNA fragments from cDNA clones. First is the issue of availability. Good cDNA samples are more cumbersome to procure. In cDNA samples procured commercially, about 30 percent of the clones contain inaccurate or wrong identities, which makes them not useful and difficult, if not impossible, to amplify by polymerase chain reaction (PCR). Hence, one is forced to order multiple clones for a single gene. This is not cost effective and can lead to experimental errors. Further, many genetic clones are not available commercially. It is estimated that expressed-sequence tag (EST) clones represent less than about 80% mammalian genes. Second, the entire sequence for clones having inserts that are longer than about 500 base pairs (bp) in size is often unknown. It is likely that some chimeric and/or large-intron-containing fragments may be introduced into these sequences. This is problematic, since one segment may contain sequences from two different genes, which could result in misleading data and lead to wrong interpretations. The resulting difference in size between individual cDNA fragments could be over 5-fold. This amount of deviation can produce unacceptable degrees of variation in the experimental data. Third, a high level of background signal can result since all EST sequences contain poly-adenine (poly-A), which can bring about increased levels of false hybridization and is detrimental for detection.

An alternative approach to amplified cDNA fragments uses reverse transcription (RT) products of messenger RNA (mRNA) as templates for polymerase chain reaction (PCR), i.e., RT-PCR. The problem with this approach, however, is that only about 10% to 20% of genes are expressed in a given cell or tissue type. To amplify cDNA fragments for all genes, a comprehensive collection of mRNAs from various cells or tissues and different stages of development is a must. This kind of comprehensive collection is very difficulty to obtain given current technology. In addition, this approach is severely limited in its potential to study unclonable sequences. Hence, a need exists for a new method that can amplify all kinds of gene sequences, both known and hypothetical.

### SUMMARY OF THE INVENTION

The present invention addresses the need for a simpler, yet more efficient method of amplifying gene sequences in mammalian and/or higher-order plant species. The method provides a means for large-scale production of genomic DNA (gDNA) sequences. The method comprises several steps. First, a 3′UTR of a gDNA sequence based on the presence of a stop codon and a polyadenylation signal in the gDNA sequence corresponding to an expressed mRNA sequence is identified. Alternatively, a “hypothetical” whole or partial exon from a gene defined by computer software can also be used. A predetermined gDNA sequence within the 3′UTR is then selected, preferably using computer software. The predetermined gDNA sequence has an overall homology of less than or equal to about 40% to any other genomic sequence in the same genome. A probe for the predetermined gDNA sequence is designed. Next, a first polymerase chain reaction (PCR) of the 3′UTR on gDNA to generate PCR-product is performed, followed by segregating the resultant PCR-product by a size-separation process selected from the group consisting of electrophoresis and chromatography. The predetermined gDNA sequence within the 3′UTR has a length of about 200 to about 600 nucleotide bases. A predetermined band from the size-differentiated samples is chosen, and a second polymerase chain reaction is performed to amplify the sample. The method can generate large quantities of gDNA probes, which enables greater efficiency for printing in microarray formats.

The present invention also includes a biological array. The biological array comprises a substrate and deposited on the substrate a set of amplified gDNA fragment sequences generated according to the method above. Each amplified sequence is derived from the sequence of at least one exon, or a partial exon, and contains no polyadenosine nor requires a vector sequence.

Additional features and advantages of the present invention will be disclosed in the detail description that follows.

### BRIEF DESCRIPTION OF THE DRAWINGS

FIG. 1 is a schematic that illustrates the 3′UTR of a gene defined by the presence of a translational stop codon and polyadenylation (polyA) signal, as well as its relative location on the human genome. The boxes, on the left, represent exons. The longer open box, on the right, represents the last exon containing the 3′UTR. GSP stands for gene specific primer.

FIG. 2 is a flowchart to demonstrate how to define a unique sequence within the 3′UTR of a gene and design a pair of primers for PCR amplification of the sequence directly from genomic DNA.

FIG. 3 is a schematic representation of a flowchart for PCR amplifications. The basic steps are listed along the center. The schematic at left shows the strategy using T7/T3 primers for the second PCR, while the schematic at right shows the strategy using gene specific primers (GSP) for both rounds of PCR.

FIG. 4 shows size distribution of the 3′UTR for 117 genes. Genes are classified along the X-axis into three groups based on the size of their 3′UTR: 1) <200 bp, 2) between 200 to 400 bp, 3) >400 bp. The number within each bar represents the number of genes within each group (Y-axis).

FIG. 5A is an image of an agarose gel of the PCR products from the first round for 12 genes. The number of each sample is indicated along the top, and flanked on each side by a molecular weight marker graded in increments of 100 bp (ladder). The 600 bp band is indicated by a line with an arrow head.

FIG. 5B is another image of an agarose gel of the PCR products from the second round for 24 genes. The number of each sample is indicated along the top. The molecular weight marker in increments of 100 bp (ladder) is shown at right. A line with an arrowhead indicates the 600-bp band.

### DETAILED DESCRIPTION OF THE INVENTION

### Definitions

The term “alternatively spliced messages,” as used in the context of the present invention, refers to mature mRNAs originating from a single gene with variations in the number and/or identity of exons, introns, and/or intron-exon junctions.

The term “biosite” as used herein means a discrete area, spot or site on the active surface of an array, or base material, comprising at least one kind of immobilized biological material for use as a probe or other functionality.

The term “chimeric,” as used in the context of the present invention, describes genes or constructs wherein at least two of the elements of the gene or construct—such as a sequence from one gene linked or physically connected with a sequence from another gene—are heterologous to each other.

The term “gene,” as used in the context of the present invention, encompasses all regulatory and coding sequences contiguously associated with a single hereditary unit with a genetic function. Genes comprise exons (coding sequences) that may be interrupted by introns (non-coding sequences). Genes can include non-coding sequences that modulate the genetic function, which includes, but is not limited to, those that specify polyadenylation, transcription regulation, DNA conformation, chromatin conformation, extent and position of base methylation and binding sites of proteins that control all of these. A gene's genetic function may require only RNA expression or protein production, or may only require binding of proteins and/or nucleic acids without associated expression.

The term “gene family,” as used in the context of the present invention, refers to a group of functionally related genes, each of which encodes a separate protein.

The term “heterologous sequence,” as used herein, refers to genetic sequences that are not operatively linked, or in nature are not contiguous to each other.

The term “homologous gene” or “homologous sequence,” as used herein, refers to a gene that shares sequence similarity with the gene of interest. This similarity may be only a fragment of the sequence and often represents a functional domain, such as a DNA binding domain, a domain with tyrosine kinase activity, or the like. The functional activities of homologous genes are not necessarily the same.

The term “public sequence,” as used herein, refers to any sequence that has been deposited in a publicly accessible database. This term encompasses both amino acid and nucleotide sequences. Such sequences are publicly accessible on the websites of the National Center for Biotechnology Information (NCBI), for example in the UniGene database (http://www.ncbi.nlm.nih.gov/UniGene). The UniGene database uses accession numbers assigned by NCBI as a unique identifier for each sequence in the databases, thereby providing a non-redundant database for sequences from various databases, including GenBank, EMBL, DBBJ (DNA Database of Japan), PDB (Brookhaven Protein Data Bank) and other like databases. The Basic Local Alignment Search Tool (BLAST) database (http://www.ncbi.nlm.nih.gov/BLAST) is used for searching.

The term “regulatory sequence,” as used herein, refers to any nucleotide sequence that influences transcription or translation of initiation and rate, and stability and/or mobility of the transcript or polypeptide product. Regulatory sequences include, but are not limited to, promoters, promoter control elements, protein binding sequences, 5′ and 3′UTRs, transcription start site, termination sequence, certain sequences within a coding sequence, polyadenylation sequence, introns, etc.

The term “related sequences,” as used herein, refers to a nucleotide sequence that exhibits some degree of sequence similarity with another sequence.

The term “sequence tagged site” (STS), as used herein, refers to a short DNA sequence that has a single occurrence in the human genome and whose location and base sequence is known. Detectable by polymerase chain reaction (PCR), STSs are useful for localizing and orienting the mapping and sequence data that are reported from many different laboratories and serve as landmarks on the developing a physical map of the human genome. Many STSs are derived from bacterial artificial chromosome (BAC) and/or P1 (bacterial phage) artificial chromosome (PAC) end sequences. Expressed sequence tags (ESTs) are STSs derived from cDNAs.

The term “untranslated region” (UTR) is a contiguous series of nucleotide bases that is transcribed, but not translated during synthesis of a peptide or protein. These untranslated regions may be associated with particular functions such as increasing mRNA message stability. Examples of UTRs include, but are not limited to polyadenylation signals, termination sequences, sequences located between the transcription start site and the first exon (5′UTR) and sequences located between the last exon and the end of the mRNA (3′UTR), including regulatory sequences.

### Description

The method and devices embodying the method of the present invention circumvents the problems associated with generating cDNA fragments from DNA clones or long oligonucleotides. The present method enables one to perform large-scale amplification of expressed sequences directly from mammalian genomic DNA (gDNA) as the starting material. This feature is an advantage, since gDNA is easier to obtain than RNA for more genetic sequences. The present method generally abstains from using clonal DNA (cDNA) or RNA-derived sequences. Rather, by means of simple PCR amplifications without cloning, the method produces amplified sequences that have greater specificity and size consistency than that observed with cDNA fragments, and allows for greater signal sensitivity than oligonucleotides.

PCR amplification of expressed sequences from gDNA of prokaryotic organisms, such as bacteria, and lower-order eukaryotic organisms, such as yeast, has been a relatively simple task. This is because, at about 100-1000 times smaller than the genome of humans or other mammalian species, the genome of prokaryotes and lower-order eukaryotes are relatively simple and do not have repetitive sequences or virtually no introns. (Yeast has only three genes that are found to contain small introns.) To do PCR amplification directly from gDNA of mammalian or other higher-order eukaryotes has been traditionally either nearly impossible or fraught with great difficulties. In contrast to single cell organisms, mammalian or higher-order eukaryote genomes are much more complex, possessing many intron segments that divide gene sequences into multiple exons and many more, longer regulatory sequences. During the natural transcription and gene expression process, a precursor RNA containing both exons and introns is first transcribed. The introns are removed subsequently through splicing to form mRNA, i.e., expressed sequences. The presence of multiple introns often complicates the task for researchers to amplify coherent, accurate, expressed gene sequences by means of PCR amplification.

With PCR, it is possible to amplify a single copy of a specific target sequence in gDNA to a level detectable by several different methodologies. For instance, the methods may include hybridization with a labeled probe; incorporation of biotinylated primers followed by avidin-enzyme conjugated detection; or, incorporation of ³²P labeled deoxynucleotide triphosphates into the amplified segment. Although PCR amplification of human genomic DNA has been used to identify sequence-tagged sites (STS), simple sequence length polymorphism (SSLP), single-stranded sequence conformation polymorphism (SSCP), or single nucleotide polymorphism (SNP) when the sequence for the region of interest is available, the applications that use these kinds sequences do not need large quantities of the PCR products, as would be required in the preparation of DNA microarrays. Indeed, even though some have suggested using amplified human gDNA with primer pairs to generate STS probes, whereby selected primer pairs corresponding to the 3′UTR of gene transcripts are employed, it is doubtful that they can generate sufficient amounts of amplified product. This is so because of two basic factors. One, primers adapted from STS do not have the specificity designed for gDNA amplification, which can not effectively control for the guanine-cytosine (G-C) content or overall quality of the primers. Two, a direct use of STS from gDNA for PCR reactions raises the potential for contamination by the gDNA in the preparations, which can lead to greater background or mismatched-hybridization signal. Furthermore, a detailed methodology is lacking.

More importantly, the applications that use the kinds of sequences discussed tend to be indiscriminate about which particular sequence or region of gDNA is used; that is, these applications do not necessarily select for expressed gDNA sequences, which is a particular subpart of coding regions in a gene. Rather, expressed and non-expressed sequences alike may be mixed together with no particular specificity. For the purposes of the present invention, to amplify expressed sequences from genomic DNA is usually difficult without previous knowledge of the intron/exon boundaries for a given gene. Mammalian introns often range in size from less than about 100 to over 10,000 base pairs (bp). The distance between two exons could be too long to be amplified by a regular PCR, and one or both primers could cross the boundary of two exons. This characteristic makes it very difficult for PCR process to work.

Although no systematic study has been conducted on the genomic structure of the 3′untranslated region (3′UTR) for all known genes, numerous studies of the genomic structure for various genes indicate that the 3′UTR often exists as a single exon. Typically, the 3′UTR is the longest exon and forms part of all expressed sequences in gDNA. The 3′UTR is very specific, containing within it a unique sequence for each given gene. This phenomenon makes the 3′UTR a valuable tool to differentiate individual genes within a gene family. While not intending to be bound by theory, it is believed that one can amplify the 3′UTR from genomic DNA without having to rely on any information regarding the intron/exon boundaries. The 3′UTR can unlock the potential for high-throughput amplification of DNA sequences directly from gDNA, for the purpose of using gDNA in high volumes in the fabrication of high-density microarray products according to the present invention.

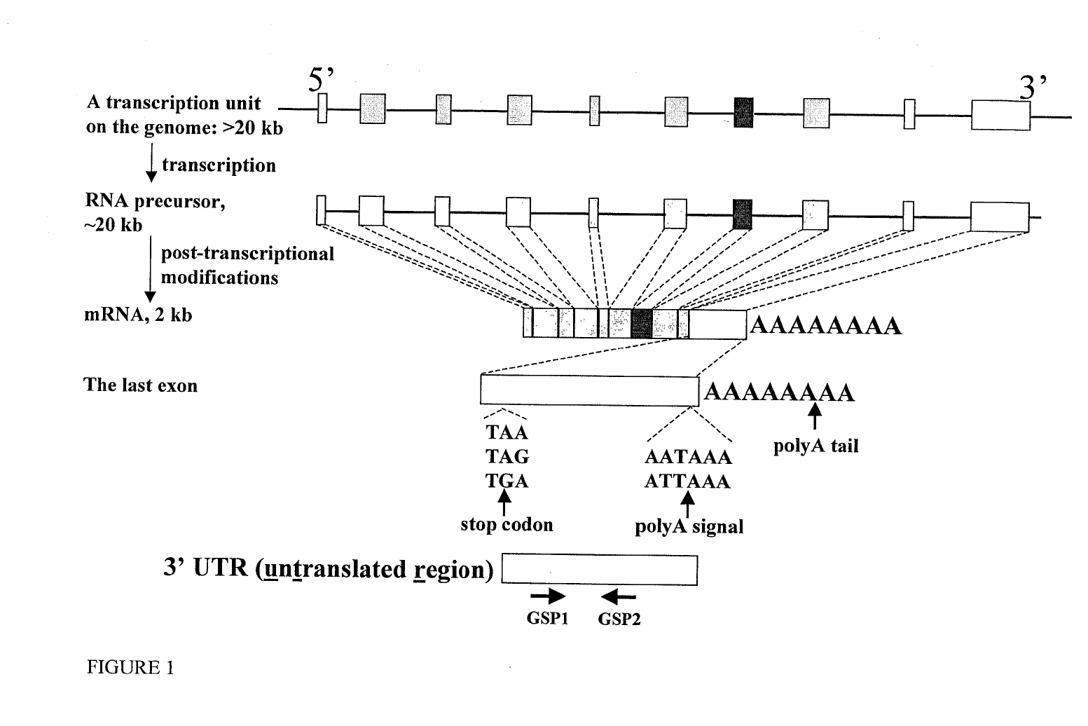

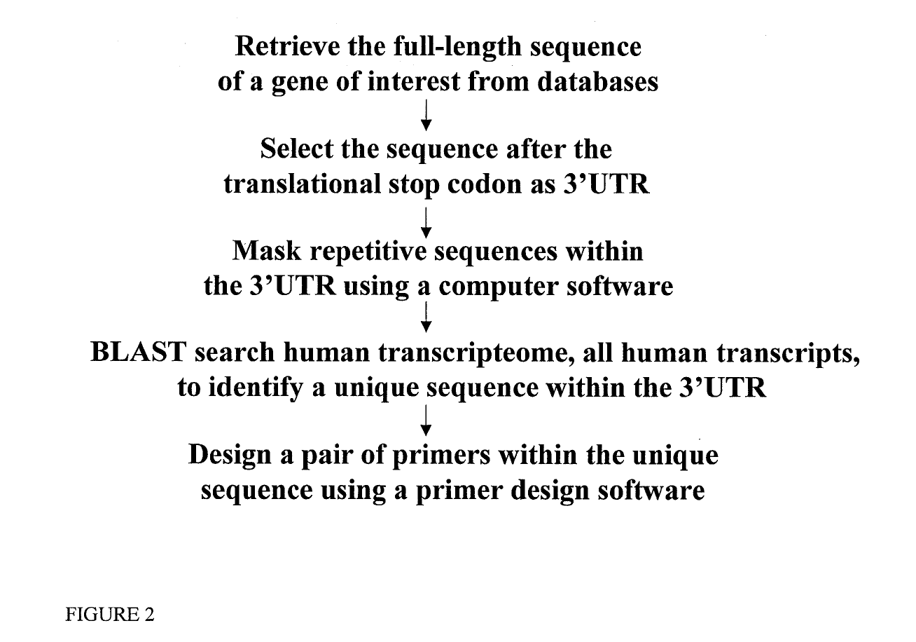

The method of the present invention, having been developed according to the principle described above, has the following protocol. First, a gene having a known public sequence is derived from a publicly accessible database, such as the UniGene database, and analyzed using a pair wise search by means of BLAST. A 3′UTR or an exon of that gene is defined or identified by the length between the translational stop codon (e.g., TAA, TGA, or TAG) and the last nucleotide before a polyadenylation signal (e.g., AATAAA or ATTAAA). For the present method to work more effectively, the 3′UTR should have a length of about at least 200 nucleotide bases. Second, a segment of sequence within the 3′UTR, ranging from about 75 to about 2000 nucleotide bases is further selected by BLAST-searching the original gene sequence against the entire UniGene database using a gene- or oligo-designer computer software program. Selected sequences have preferably about 200 nucleotide bases or less, to about 800 nucleotide bases or more. More preferably, the selected sequence has a length of about 200 bases to about 500 or 600 bases, more preferably from about 225 or 250 bases to about 400 or 450 bases. The purpose of this second step is to minimize homologous sequence that may be otherwise also selected for in the PCR process. Thus, the accuracy and efficiency of downstream PCR amplification is improved. Generally the less homologous, or more heterologous, the sequence is to other sections of the genome the better to reduce mismatches during hybridization. The homology of the segments as used herein is determined on an overall scale comparing the selected gene sequence to all other gene sequences of the genome. That is, no clustering occurs preferably in any one region, but is rather diffused throughout the sequence. The selected gDNA segment has an overall amount of homology of less than or equal to about 70% for highly homologous gene families, but is more commonly less than or equal to about 40%. Preferably, the overall homology is about 35% to about 20%-15% or less. Use of gene-designer computer software also permits one to pick the PCR segments in a high throughput mode, so that one can select segments of sequences for PCR in a large-scale and automated fashion. FIG. 1 illustrates the process described above in schematic form, and FIG. 2 further describes the process in a flow chart.

Third, a primer design software, like web-based Primer 3 (http://www.genome.wi.mit.edu/cgi-bin/primer/primer3 www.cgi), is used to design a complement for the selected or predetermined gDNA sequence. The primers in reaction, in contrast to STS probes that are spotted on a surface, are designed with greater specificity for gDNA amplification according to more stringent parameters in terms of sequence length and about 50-60% G+C content. Individual primers are verified by BLAST search for correct gene origin and absence of random overlapping sequences. Generally, the primer designed for a given segment should not contain a related sequence. Table 2 lists all primer sequences used. Two types of primer pair were designed at about 500 bp apart (or within 200-400 bp when the 3′UTR is less than 500 bp long) and away from repetitive sequences. Type I contains a T7 promoter at the 5′end of the gene specific primer (GSP) in the sense direction and a T3 promoter at the 5′end of the GSP in the anti-sense direction. In particular, the sequence for T7 promoter is 5′-TAATACGACTCACTATAGGG-3′ and for T3 promoter is 5′-ATTAACCCTCACTAAAGGGA-3′ (derived from Invitrogen™). Type II primers only contain gene specific sequences. All primers were purchased from Sigma-Genosys™ as desalted and dried pellets. Each pellet was dissolved in ddH₂O to a final concentration of 500 μM.

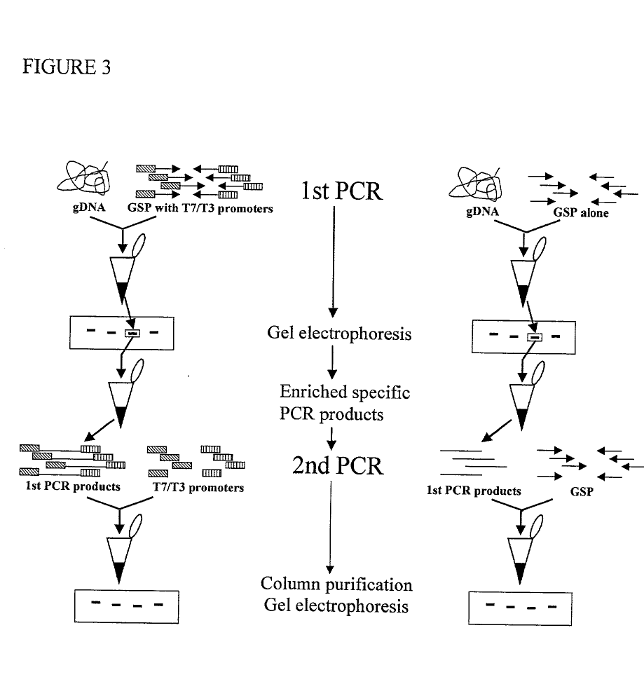

Next, a first round of PCR is preformed under predetermined conditions, which will be explained more fully in the Experiments section, below. Two different strategies were applied. As shown in FIG. 3, the flowchart, Strategy 1 is to employ Type I primers (GSP with T7 or T3 promoter at a 5′end) for the first PCR, then use T7 and T3 primers for the second PCR (FIG. 3, left panel). The other, Strategy 2, is to use the same pair of gene specific primers, Type II primers (GSP alone), for both first and second round of PCR (FIG. 3, right panel).

Generally, the PCR product from this first round are then separated according to size-differentiation. Various size-differentiation processes, such as electrophoresis or chromatography (e.g., High Performance Liquid Chromatography), may be used. The size-differentiated sequence sample or band of interest is then gathered up by a transfer pipette, without need for purification—this is, without the need to remove each sequence-band from its gel bed—and suspended in a small volume (˜50 μL) of water.

A second round of PCR is performed on the predetermined sequence sample under the same conditions as in the first round of PCR. The PCR product from this second round is subjected to column purification or gel electrophoresis to clean up the amplified sequences using a commercial purification kit and eluted into a final volume.

The final amplified sequence(s) derived according to the method can be printed or otherwise deposited as an array of biosites on a treated glass (e.g., borosilicate, aluminosilicate, fused silica, treated with a propylsilane or the like), polymer (e.g., polystyrene or polypropylene, nylon filter), or metallic (e.g., gold, platinum, chromium, or silicon) substrate for DNA micro-assay purposes. These kinds of arrays are the functional heart of DNA microarrays used in genomic studies, drug discovery, and other biological assays. The device can be characterized as having a set of gDNA fragments having the sequence of one exon having no poly-adenosine nor vector sequence, and having a sequence length that range from about at least 75-80 bases to about 1800-2000 bases. Preferred fragment lengths are about 200 to about 600 or 800 nucleotides. Particular uses and means of fabrication of specific arrays are described in detail in International Patent Application No. WO 00/77257, entitled “Gene Specific Arrays and the Use Thereof,” by Narayan Baidya et al., the complete contents of which are incorporated by reference into the present disclosure.

The DNA fragments, generated according to the present invention, function essentially like cDNA fragments that have been amplified from cDNA clones, but provide many advantages with few of the associated drawbacks. The present invention solves the procurement problem, since the method is not limited by or dependent on the availability of cDNA clones, nor does it depend on bacterial cultures. Hence, with gDNA fragments generated according to the present invention, it is possible to cover the entire mammalian genome. The method has an overall shorter processing time than current methods since it requires neither cloning nor initial purification after the first round of PCR. Using the method, one can maintain quality control relatively easily. Partially as a result of prior determination and size-differentiation, the final expressed gDNA sequences generated and amplified according to the inventive method have small size variations between individual amplified strands and no poly-adenosine sequences. This feature promotes more functional consistency in the amplified sequences. Further, in operation, they do not require vector sequences.

The method described here can be used widely to amplify expressed sequences from the genomic DNA of humans and other mammalian animals, as well as higher order plants. With the recent completion of sequencing of the entire human genome and of many other mammalian genomes, the intron/exon boundaries for all genes will soon be known. Since there is always one or multiple exons with a size longer than about 500 bp, the length of the 3′UTR will no longer be a limiting factor. All expressed sequences for virtually all genes can be amplified using this method. Even genes with currently hypothetical exons can be identified through use of the present invention. The sequence for hypothetical exons can be defined by computer software. Even though predicted by gene prediction software, many genes in these genomes, however, may not be clonable—thus, not available as cDNA clones. At present, the only way to study unclonable sequences of genes is to use synthetic oligonucleotides. The present method amplifies expressed sequences of gDNA of at minimum about 75 bases—preferably about 200 bp—or longer, providing better performance than oligonucleotides, which can not provide sufficient signal due to their limited lengths (<100-150 bp).

When amplifying expressed sequences from genomic DNA, a major issue is how to procure a sufficient amount of PCR fragments to print arrays on surfaces. The PCR amplification process is known to reach a plateau concentration of specific sequences. The human genome has about 3.2 billion base pairs. The amount of unique 1000 bp sequence within 10 μg of total genomic DNA is estimated to be about 0.32 pg. A single run of a single PCR reaction under a standard condition, i.e., to use 1 μg genomic DNA in 50 μL reaction for 35 cycles, usually yields less than 1 μg of PCR product at most. Multiple reactions will consume great amounts of gDNA, which is quite expensive. Hence, a second round of PCR is usually necessary to secure a sufficiently large quantity. Performing a second round of PCR using the first PCR product as templates directly, without purification, however, traditionally results in high background, which are seen as a big smear around the specific PCR product, as mentioned above. This phenomenon suggests that the presence of irrelevant sequences that may cause researchers to misinterpret the data from subsequent array analysis.

The present method alleviates these problems—minimizing, if not eliminating them—through several advantageous features. It is believed that due partially to size-differentiation and one or more second round(s) of PCR, the present invention can produce at least about twice—if not three to five times or more—the amount of amplified product than that which can be attained through use of other ways of generating probes. The strands of amplified sequences generated using the present method are relatively size constant. Moreover, because gDNA does not contain polyadenosine sequences, nor undergoes polyadenylation, which is a post-transcriptional process, there is little likelihood of false hybridization. Since there is no poly-A to remove, the method saves time in the process.

The most commonly used protocol, currently available, to generate large amounts of gene specific PCR products is to perform a so-called nested PCR. That is, perform a first round of PCR with a pair of GSPs, and then a second round of PCR using another pair of internal GSPs. According to this procedure, each gene needs four GSPs for the PCR. The protocol, thus, creates more work in the design of the primer and also doubles the cost. This means that researchers need to design two pairs of primers, which is a possible limitation to the process. It is difficult to find a second pair of primers within the segment defined by the first primer pair.

An approach, practiced in small scale laboratory work, is to perform a first round of PCR, cut-out a gel slice containing the products from the first PCR, purify the DNA using commercially available kits, and then use it as templates for the second round of PCR. This process, however, is time consuming. The inventive method eliminates the need for a purification step, which is one of its important improvements over the prior art, and enables large-scale production of large amounts of amplified sequence in a high-through-put manner for DNA microarrays. Instead of using a laser bladder to cut individual DNA bands out of the gel for purifying, the present inventive method permits the user to simply pick DNA, together with agarose, out of the gel using a transfer pipette and soak the DNA in ddH₂O (about 50 μL) without purification. The DNA eluted from the agarose is sufficient for about at least 50 second-round polymerase chain reactions. Small amount of second PCR products can be saved when diluted in a large volume of buffer for a lifetime supply. A follow-up sequence identity check can usually confirm a product and remove any concerns about nonspecific PCR products or related sequences having similar size of the gene-specific products mixed together in the final products.

As mentioned before, two strategies are to be applied for amplification of the 3′UTR. The first strategy employs GSP with T7/T3 promoters for the first PCR, then use T7/T3 for the second PCR. An advantage of the first strategy is that it is able to simplify the procedures for a second round of PCR and subsequent sequencing verification of the final PCR products, because only a single pair of universal primers is required. Another advantage is having T7 and T3 promoters at both ends. Researchers will be able to generate RNA in either a sense or anti-sense direction, which ever and whenever necessary. The second strategy employs the same GSPs for both first and second rounds of PCR. This approach has several advantages. It simplifies primer design, cuts the cost, and can avoid cross contamination problems. Additionally, the second strategy enables better verification of sequence, which provides a means for quality control of second-round PCR products since no PCR product will be generated if a mistake was made in mixing templates with primer pairs. No such control, however, will be associated with the first strategy because the universal primers can amplify any sequences from the first round of PCR.

### Experiments

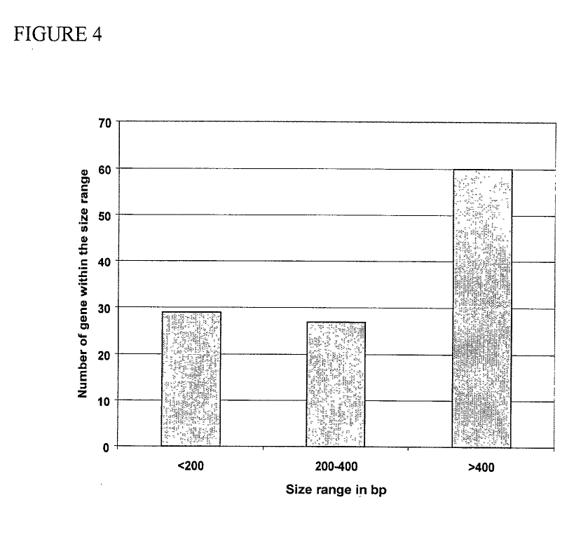

Experimental studies were conducted for 117 genes using the present method for amplifying expressed sequences from human genomic DNA. First, the relative size-distribution of the 3′UTR was ascertained according to the steps described above. The sequences for 117 putative tox genes were retrieved from the UniGene database and their respective 3′UTR were defined to determine how many genes have a 3′UTR length sufficient for PCR amplification. As shown in FIG. 4, the 3′UTR for 29 genes are shorter than 200 bp (˜24%), for 27 genes are between 200 to 400 bp (23%), and for 60 genes are over 400 bp (51%). Although the method can work with sequences of considerably less than 200 bp, such as short as 75-100 bp, a practical, minimal length required for PCR is about 200 bp. About 74% genes can be potentially amplified. Considering the constraints on sequence contents for primer design, 97 genes, each having a 3′UTR over 400 bp, were selected for PCR amplifications.

Overall, two rounds of PCR were necessary to obtain sufficient DNA for array printing. The first round of PCR was carried out in a 10 μL reaction volume under following conditions. Reagents: 1× buffer containing 1.5 mM MgCl₂ (PE Biosystems), 0.2 mM dNTP (GIBCO BRL), 0.4 μM of each primer, 100 ng human placenta genomic DNA, and 0.5 units of Taq polymerase (Roche Molecular Biochemicals). PCR cycles: one cycle of 95° C. for 1 minute, 25 cycles of 94° C. for 30 sec., 60° C. for 30 sec., and 72° C. for 45 sec., and one cycle of 72° C. for 5 minutes. Gel electrophoresis was used to size-differentiate the PCR product on a 1.5% agarose gel. A transfer pipette picks up the DNA band with the expected size as defined by primer design software together with the slice of the gel on which the DNA rested, and placed the DNA in 50 μL water to soak. One microliter of the DNA eluted out of the gel slice was used as templates for second round of PCR using either T7/T3 primers or GSPs in 50 μL reaction (8 reactions per gene) under the same condition described above. The PCR products (in total volume of 600 1 for each gene) were cleaned using QIAquick PCR Purification kit (Qiagen), and eluted in a final volume of 100 μL. One microliter of each product was loaded on a 1.5% agarose gel for verifying sizes and estimating concentrations. A randomly selected set of DNA samples was measured for OD₂₆₀ to set a standard for the adjustment of the DNA concentration for all PCR products.

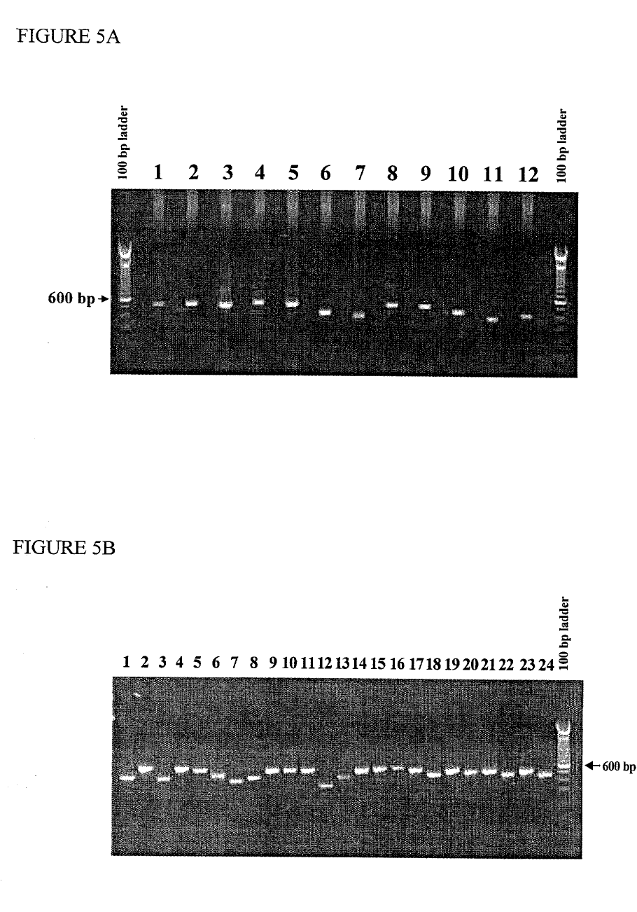

The results from the first round of PCR amplification are shown in FIG. 5A. Twelve genes were selected as proof of concept examples from the original 97 genes. The PCR products for the 12 genes that were amplified using Type I primers produced distinct, unique bands, each with the expected size. PCR, although a good tool, is still not sufficiently specific, nor perfect in amplifying correct sequences. The faint smear present in each lane of the gel represented nonspecific PCR products. Size-differentiation by gel electrophoresis, for instance, removes extraneous strands of a wrong sequence length. The wide DNA band observed near the loading well was from input genomic DNA. To remove nonspecific PCR products, a gel slice containing the DNA band of interest with correct length was removed and transferred to a tube containing about 50 μL of ddH₂O. The DNA eluted from the gel slice was then used for a second round of PCR. After electrophoresis column purification, 1 μL of each PCR product was again loaded on a gel for electrophoresis. FIG. 5B shows the results from a second round of PCR using another 24 genes, also selected as examples of the original 97 genes, amplified using Type II primers. As seen in FIG. 5B, all PCR products for the 24 genes gave a distinct single band, without visible background. All 12 genes amplified using the Type I primers, shown in FIG. 5A, also gave the same results (data not shown). Generally, it was observed that once the first round of PCR amplification was done successfully, the second round of PCR would always work well, regardless the variations of the yield from gene to gene during the first round of PCR. In this particular experiment, over 90 percent of PCR products contained the correct sequence. In the field of microarray fabrication, an overall correct result of as high as over 90% is generally regarded as an excellent success rate for generating printable nucleic acid materials—especially in view of the difficulty of amplifying the kinds of genes selected herein.

Table 1 summarizes the results observed for both PCR products and sequencing. As recorded in Table 1, upper panel, a total of 97 genes were tried for PCR amplification. In the first round, the PCR products for 95 genes (95%) exhibited a distinct single band with their respective, expected size, and two genes (˜2%)—BRAC2 (>900 bp) and CASP2 (>1200 bp)—had a single product longer than the cDNA sequence. The PCR products for three genes (˜3%)—CASP13, COX11 and USP6—had multiple bands from which no specific product could be identified. All PCR products were sequenced through the service provided by SeqWright Inc. Samples were prepared following manufacturer's instructions. Briefly, individual PCR products were diluted in ddH₂O to a final concentration of 50 ng/μL, and sequencing primers to 3.2 μM. The PCR products with either the correct size or wrong size for 94 genes were sequenced using a primer from sense direction. The results were summarized in the lower panel of Table 1. Briefly, the PCR products for 85 genes contain the correct sequences (90%); the sequences for 7 genes were not readable due to the presence of mixed sequences; and there were no signal for 2 genes probably due to sequencing system error (2%).

TABLE 1
Summary of Results Observed for PCR Products and Sequencing
Gene Numbers (PCR)

| Total | With expected size | With wrong size | No specific product |
|---|---|---|---|
| 97 | 92 | 2 | 3 |
| | (95%) | (2%) | (3%) |

Gene Numbers (Sequencing)

| Total | With correct sequence | Not readable | No signal |
|---|---|---|---|
| 94 | 85 | 7 | 2 |
| | (90%) | (8%) | (2%) |

The top panel shows the results of the first PCR; the bottom panel shows the results of sequencing. The percentile within parenthesis is calculated as follows: the number of genes within each category divided by the total number of genes shown in the first column.

Although the present invention has been described in detail, persons skilled in the art will understand that the invention is not limited to the embodiments specifically disclosed, and that various modification and variations can be made without departing from the spirit and scope of the invention. Therefore, unless changes otherwise depart from the scope of the invention as defined by the following claims, they should be construed as included herein.

TABLE 2

| Symbol | Accesion No. | Sense primer | Antisense primer | Expected size, bp |
|---|---|---|---|---|
| AATK | NM_004920 | AATKs: T7-cttcactgactcagctagac* | AATKa: T3-accagcgttctaagcctcaa* | 516 |
| ABCD3 | NM_002858 | ABCD3s: T7-tgactccaggaaaagccatt | ABCD3a: T3-tcgcttaggatcgtttgaca | 537 |
| ABCB10 | NM_012089 | ABCB10s: gcatggcacctcattttctt | ABCB10a: T3-agcagtwatgccttgcttc | 484 |
| ABCF1 | AF027302 | ABCF1s: atcccactctgattgcatcc | ABCF1a; gttcagcagcattcctttcc | 408 |
| ACTB | NM_001101 | ACTBs: T7-tgcgttacaccctttcttga | ACTBa: T3-gggagaccaaaagccttcat | 541 |
| ADH2 | NM_000668 | ADH2s: T7-gggccattgtgattgaagtc | ADH2a: T3-cattcacagcatttgccatc | 559 |
| AMPH | NM_001635 | AMPHs: T7-ccctgcagaagatgtgatga | AMPHa: T3-tagcctacctccagccacag | 540 |
| ANXA5 | NM_001154 | ANXA5s: T7-gcautgtatgccagtgctt | ANXA5a: T3-ttcagggggacagaaatgtt | 441 |
| AOC3 | NM_003734 | AOC3s: T7-ccagagtagggttgccagtc | AOC3a: T3-attatcattgcacccccaaa | 540 |
| API4 | NM_001168 | API4s: T7-caggtgcctgttgaatctga | API4a: T3-aaggttgggctgacagacac | 539 |
| ATF3 | NM_001674 | ATF3s: T7-ccagggttgtgctttctagc | ATF3a: T3-ctggtaccaccagctccact | 527 |
| BAD | AF021792 | BADs: T7-agtgaccttcgctccacatc | BADa: T3-cagacgcgggctttattaac | 417 |
| BCL2 | NM_000633 | BCL2As: T7-tggtgggaggaaaagagttg | BCL2Aa: T3-tctgagctccatcagcttcc | 538 |
| BID | NM_001196 | BIDs: gaacggacagttccagaag | BIDa: tggaaataaaggcaccgtgt | 293 |
| BRCA2 | NM_000059 | BRCA2s: T7-catttgcaaaggcgacaata | BRCA2a: T3-ctcaagtttgagtttggatgac | 533 |
| CALR | NM_004343 | CALRs: gcgccaaataatgtctctgtg | CALRa: agaaagggaggggtgaaatg | 406 |
| CASP2 | NM_001224 | CASP2s: gactgatcgtggggttgac | CASP2a: agaacagaaaccgtgcatcc | 482 |
| CASP3 | NM_004346 | CASP3s: catggtcaaaggctcaaacc | CASP3a: catgtctctgctcaggctca | 528 |
| CASP6 | NM_001226 | CASP6s: ccaggcgtggttactcaca | CASP6a: ccatggccaacatgaacttt | 427 |
| CASP7 | NM_001227 | CASP7s: tccactgcaattggtggtaa | CASP7a: tggctttgttcttgtcatgg | 500 |
| CASP10 | NM_001230 | CASP10s: caggcaaagcttgaatcagg | CASP10a: cacctggctgaagtcaaatc | 509 |
| CASP13 | NM_003723 | CASP13s: cagggtgaaaggagatggtg | CASP13a: aagtggtacatctccttagtc | 497 |
| CAT | NM_001752 | CATs: taacccgctcatcacrggat | CATa: attaagccatgacggtgctc | 445 |
| CCNC | NM_005190 | CCNCs: aaacattccgaagaattcca | CCNCa: ggtccctcaatgaccaaaga | 376 |
| CCNE1 | M74093 | CCNE1s: ccatccttctccaccaaaga | CCNE1a: ctatgggctctgcacaacg | 403 |
| CCNF | NM_001761 | CCNFs: gctgccatccacttctgttt | CCNFa: ggtggccagaattcccttat | 501 |
| CCNG2 | NM_004354 | CCNG2s: agccatcaaatggggtagtg | CCNG2a: cttggggcaataggaatgaa | 501 |
| CDC10 | NM_001788 | CDC10s: caaaggttccattcagtgcag | CDC10a: cttcaagaggccatgattcc | 491 |
| CDC23 | NM_004661 | CDC23s: gaccttgctcttggatttgc | CDC23a: acaggcctgaaactctccaa | 505 |
| CDC25C | NM_001790 | CDC25Cs: ggctgctaacaagtcaccaaa | CDC25Ca: caacgctcttgcatagccc | 324 |
| CDC37 | NM_007065 | CDC37s: ctgcttccagcccctatgt | CDC37s: gacacagacagcagacgaaca | 340 |
| CDC42 | NM_001791 | CDC42s: gacaaatgccctgcacctac | CDC42a: caatccgtcctcctcccua | 422 |
| CDKN2A | NM_000077 | CDKN2As: tctgagaaacctcgggaaac | CDKN2Aa: gccatttgctagcagtgtga | 414 |
| CHES1 | NM_005197 | CHES1s: cctccagcttgtcagaaacc | CHES1a: gccaatcttcaggcttatgg | 501 |
| CLGN | NM_004362 | CLGNs: agcatgccagacctgaacn | CLGNa: tgaacaaggcatgtccttaaa | 520 |
| COX10 | NM_001303 | COX10s: gtgagcctcatgatctgctg | COX10a: ccagcacacccttcuccta | 502 |
| COX11 | NM_004375 | COX11s tcacgctgttgtcaggaatc | COX11a: attctttaggggccaggatc | 480 |
| COX15 | NM_004376 | COX15s: tgaccccatcgagatgaaat | COX15a: cagctctgcagcataatgga | 496 |
| CPT2 | NM_000098 | CPT2s: gctaccatcacttcctcatc | CPT2a: tttccaaacctttcctcctg | 524 |
| CYP1B1 | NM_000104 | CYP1B1s: tggggacagaactcccatta | CYP1B1a: ccatgctttgaattttgtgc | 509 |
| CYP3A3 | NM_000776 | CYP3A3s: gcctgagaacaccagagacc | CYP3A3a: tgtcattgttagagccatcaaaa | 320 |
| CYP4A11 | NM_000778 | CYP4A11s: cctgtctgcccatatcctgt | CYP4A11a: tgtgacggtttagcatctgc | 499 |
| CYP4B1 | NM_000779 | CYP4B1s: atgagaatggggtcccagat | CYP4B1a: catctcagtgaaggggcact | 426 |
| CYP4F2 | NM_001082 | CYP4F2s: ccctaagaccctgttccaca | CYP4F2a: gtgtcgtgctaccttcgtca | 492 |
| CYP4F3 | NM_000896 | CYP4F3s: cccactaaaatgacccctga | CYP4F3a: tcaccatcccaggagaaaac | 497 |
| CYP7A1 | NM_000780 | CYP7A1s: ttgttcaccagtgcttgctt | CYP7A1a: atgatcacacccgaagaacc | 499 |
| CYP7B1 | NM_004820 | CYP7B1s: ccctaaacatcctaagctcatct | CYP7B1a: gggaaacattttcatccagtg | 439 |
| CYP8B1 | AF090320 | CYP8B1s: cttctatccccagacccac | CYP8B1a: ttggagaaagctggcaaagtt | 500 |
| CYP19 | NM_000103 | CYP19s: ccaaacccacctgctagtgt | CYP19a: cccccaatcactgtagctgt | 506 |
| CYP24 | NM_000782 | CYP24s: tgggatccaaggcattctac | CYP24a: caaataatgccccagtgaatc | 510 |
| CYP51 | NM_000786 | CYP51s: actcatcgctcttgccaaat | CYP51a: gaagcagggaacaactgagc | 503 |
| DAPK3 | NM_001348 | DAPK3s: gggctgcttctctacacagc | DAPK3a: atttctcttggctgcagagg | 443 |
| DHFR | NM_000791 | DHFRs: gggaacagtgaatgccaaac | DHFRa: atgcaaccctttggttcaag | 499 |
| DNJ3 | NM_004222 | DNJ3s: ctgcaaacaaattgcacagg | DNJ3a: gccaaacacaaagcucagg | 385 |
| DPYD | NM_000110 | DPYDs: cccttcgctgaaattgctta | DPYDa: tgaagatgccatgaagagga | 481 |
| DTR | NM_001945 | DTRs: cctttgccacaaagctagga | DTRa: cagctccaatguccctgtt | 493 |
| EGF | NM_001963 | EGFs: caaattgggacaacagtgctt | EGFa: tgtgcaatcacaccaagagg | 461 |
| EGR1 | NM_001964 | EGR1s: ccttgctcccttcaatgcta | EGR1a: catgtccctcacaattgcac | 501 |
| EPO | NM_000799 | EPOs: ctccctcaccaacattgctt | EPOa: gtcttcatggucccaccac | 453 |
| FADD | NM_003824 | FADDs: tgcgggagtagttggaaagt | FADDa: ttgcaggacccataatcctc | 506 |
| G6PD | NM_000402 | G6PDs: ttgacctcagctgcacauc | G6PDa: tagcagagaggctgcctacg | 455 |
| G17 | NM_006841 | G17s: ctaccctgctaggctctgg | G17a: cctgtttcttctcccagcag | 505 |
| GAS11 | NM_001481 | GAS11s: gaatggacagctttgcaggt | GAS11a: ctctgggcctaacctcactg | 500 |
| HIF1A | NM_001530 | HIF1As: gtggtagccacaattgcaca | HIF1Aa: gcgacaaagtgcataaaatcaa | 523 |
| HPRT1 | NM_000194 | HPRT1s: agttctgtggccatctgcu | HPRT1a: gggaactgctgacaaagattc | 483 |
| HSD11B2 | NM_000196 | HSD11B2s: cattacgatcccccaagtgt | HSD11B2a: tgtggcaattgggaagtaca | 437 |
| IER3 | NM_003897 | IER3s: gacttccgaggcaacttgaa | IER3a: cgccgaagtctcacacagua | 485 |
| IGF2R | NM_000876 | IGF2Rs: attcgaagaaacccttgctg | IGF2Ra: atctttgggcaggugtttg | 506 |
| ITGA5 | NM_002205 | ITGA5s: gaagcctttgcattttggag | ITGA5a: ggaaattcctggcttctcct | 493 |
| LPL | NM_000237 | LPLs: tatagctgggaacccgactg | LPLa: gccacaatgacctttccaat | 506 |
| MADD | NM_003682 | MADDs: accggttatgtgtccctctg | MADDa: cgaccactccatcctctgat | 507 |
| MADH2 | NM_005901 | MADH2s: caatcaagtcccatggaaaag | MADH2a: atcaagaagcagcgcacac | 397 |
| MAOB | NM_000898 | MAOBs: ttccaagtttattgccctcaa | MAOBa: agacacaccgcacaaaacag | 504 |
| MAP3K8 | NM_005204 | MAP3K8s: gtgaatggtgccattttcg | MAP3K8a: tcactagtggccgtctgtca | 501 |
| MMP14 | NM_004995 | MMP14s: gggaacttccaaggaaggag | MMP14a: tcgtttgtgtgccttctctg | 499 |
| NAT2 | NM_000015 | NAT2s: ccttgtgtatgtatcacccaactc | NAT2a: agcatgaatcactctgcttcc | 243 |
| NOD1 | NM_006092 | NOD1s: tcattccaacacctgccata | NOD1a: ccatgccctatuctttgga | 502 |
| NR1I2/SXR | AJ009937 | NR1I2s: cacatacccacgtttgttcg | NR1I2a: tgcccttgctcctacagact | 506 |
| PDCD1 | NM_005018 | PDCD1s: cagctccctgaatctctgct | PDCD1a: ggaccgtaggatgtccctct | 500 |
| RAD9 | NM_004584 | RAD9s: tgaaggctgaaccaagaacc | RAD9a: agcgccaaagagtatcagga | 495 |
| RB1 | NM_000321 | RB1s: tgaggatctcaggaccttgg | RB1a: gtgaatgggcagtcaatcaa | 486 |
| REQ | NM_006268 | RAQs: cactcttacggtcggtctcc | RAQa: tcaactccaaagcgacagtg | 496 |
| SLC15A1 | NM_005073 | SLC15A1s: ttctaagcagccagcagtga | SLC15A1a: tcattactcggccttcacct | 411 |
| SLC20A2 | NM_006749 | SLC20A2s: gcaaacagctaaagggatgg | SLC20A2a: ggttgcctgttctgaagctc | 480 |
| SLC29A1 | NM_004955 | SLC29A1s: ggtgatcctgagtggtctgg | SLC29A1a: aaggcacctggtttctgtca | 506 |
| SMAC | NM_019887 | SMACs: tgtctgtgcaccgagaagag | SMACa: cctgttg~gagcaccaggta | 505 |
| TNFRSF6/FAS | NM_000043 | TNFRSF6s: tagagctttgccacctctcc | TNFRSF6a: ggtgguccaggtatctgct | 506 |
| TNFSF6 | NM_000639 | TNFSF6s: tgttacaggcaccgagaatg | TNFSF6a: gttagtucaccgatggctc | 488 |
| TP53 | NM_000546 | TP53s: cccttgcttgcaataggtgt | TP53a: tacctaaccagctgcccaac | 502 |
| UCH37 | NM_015984 | UCH37s: gcttetgcacatattttcatgg | UCH37a: tcactggaaattatacttttgtccut | 510 |
| UGT1A1 | NM_000463 | UGT1A1s: taatcagccccagagtgcu | UGT1A1a: acaccacccaccaatttcat | 480 |
| USP5 | NM_003481 | USP5s: cttaccaatgagggcaggg | USP5a: ggcatttccagagaaggaca | 503 |
| USP6 | NM_004505 | USP6s: taatagcagcccacggacu | USP6a: ggcagagtcggtgtcaarn | 505 |
| USP8 | NM_005154 | USP8s: aggacagtgggagctgtgtt | USP8a: atacagcccaaagccaacag | 477 |
| USP11 | NM_004651 | USP11s: cctctctgcaatctcgcuc | USP11a: gggagcagactggtgcuta | 357 |
| USP14 | NM_005151 | USP14s: cacccaagattcageagtca | USP14a: gtcttcagccaagctccaac | 490 |
| USP15 | AF106069 | USP15s: gacactttcctgctggtggt | USP15a: cggggataaatttgaaaatgc | 500 |

*Type I primers with T7 or T3 promoter at the 5′ end.

We claim:

1. A method for amplifying expressed genetic sequences from gDNA selected from a mammalian or higher order plant species, for printing on DNA microarrays, the method comprises:
identifying either 1) a 3′UTR of a gDNA sequence based on the presence of a stop codon and a polyadenylation signal in the gDNA sequence corresponding to an expressed mRNA sequence, or 2) an exon of a gene defined by computer software;
selecting a predetermined gDNA sequence within the 3′UTR or exon;
designing a probe for said predetermined gDNA sequence;
performing a first polymerase chain reaction (PCR) for the 3′UTR or exon on gDNA to generate PCR-product;
separating the resultant PCR-product by a size-differentiation process selected from the group consisting of electrophoresis and chromatography;
selecting a predetermined band from the size-differentiated samples; and
performing a second polymerase chain reaction to amplify predetermined sequence;

2. The method according to claim 1, wherein a plurality of said final amplified sequences are deposited on a substrate in an array.

3. The method according to claim 1, wherein said final amplified sequences are the sequence of one exon and contains no polyadenosine.

4. The method according to claim 1, wherein said predetermined gDNA sequence within the 3′UTR or exon is selected by use of computer software.

5. The method according to claim 1, wherein said selected predetermined gDNA sequence within the 3′UTR or exon has a length of about 75 to about 2000 bases.

6. The method according to claim 5, wherein said selected predetermined gDNA sequence has a length of about 200 to about 600 bases.

7. The method according to claim 6, wherein said selected predetermined gDNA sequence has a length of about 250 to about 450 bases.

8. The method according to claim 1, wherein said selected predetermined gDNA sequence has an overall homology of less than or equal to about 70% to any other genomic sequence in the same genome.

9. The method according to claim 8, wherein said selected predetermined gDNA sequence has an overall homology of less than or equal to about 40% to any other genomic sequence in the same genome.

10. The method according to claim 8, wherein said selected predetermined gDNA sequence has an overall homology of from about 20% to 30% to any other genomic sequence in the same genome.

11. The method according to claim 1, wherein said method can generate PCR products that contain over 90 percent correct predetermined sequence.

12. The method according to claim 1, wherein said array is a rectilinear format.

13. An biological analysis device comprising a substrate and an array of a set of expressed genetic sequences from gDNA selected from a mammalian or higher order plant species located on the substrate, wherein the genetic sequences are generated according to a method that comprises:
either 1) a 3′UTR of a gDNA sequence based on the presence of a stop codon and a polyadenylation signal in the gDNA sequence corresponding to an expressed mRNA sequence, or 2) an exon of a gene defined by computer software;
selecting a predetermined gDNA sequence within the 3′UTR or exon;
designing a probe for said predetermined gDNA sequence;
performing a first polymerase chain reaction (PCR) for the 3′UTR or exon on gDNA to generate PCR-product;
separating the resultant PCR-product by a size-differentiation process selected from the group consisting of electrophoresis and chromatography;
selecting a predetermined band from the size-differentiated samples; and
performing a second polymerase chain reaction to amplify predetermined sequence.

14. The device according to claim 13, wherein said expressed sequences are printed onto said substrate.

15. The device according to claim 13, wherein said expressed sequences are arranged in a rectilinear array.

16. The device according to claim 13, wherein said selected predetermined gDNA sequence within the 3′UTR or exon has a length of about 75 to about 2000 nucleotides.

17. The device according to claim 13, wherein said selected predetermined gDNA sequence has a length of about 200 to about 600 nucleotides.

18. The device according to claim 17, wherein said selected predetermined gDNA sequence has a length of about 250 to about 450 nucleotides.

19. The device according to claim 13, wherein said amplified sequences are the sequence of at least one exon and contains no polyadenosine or vector sequence.

20. The device according to claim 13, wherein said substrate is made of a material selected from the group consisting of glass, polymer, or metallic surfaces.

21. A DNA high-density microarray comprising: a substrate upon which are deposited an array of biosites of genomic DNA fragments having the sequence of at least one exon, and absent polyadenine and vector sequences, said genomic DNA fragments having a sequence length of from about 75 to about 2000 nucleotides.

22. The microarray according to claim 21, wherein said gDNA fragments have a sequence complementary to a 3′UTR of a gene.

23. The microarray according to claim 21, wherein said gDNA fragments have a sequence of a hypothetical exon.

24. The microarray according to claim 21, wherein said gDNA fragments have a sequence of a partial exon.

25. The microarray according to claim 21, wherein said selected predetermined gDNA sequence has a length of about 200 to about 800 nucleotides.

26. The microarray according to claim 21, wherein said substrate is made of a material selected from the group consisting of glass, polymer, or metal.
